(19) United States
(12) Patent Application Publication
(10) Pub. No.: US 2017/0152468 A1
(43) Pub. Date: Jun. 1, 2017

(54) BIODIGESTION REACTOR, SYSTEM INCLUDING THE REACTOR, AND METHODS OF USING SAME

(71) Applicant: reNature, Inc., Tempe, AZ (US)

(72) Inventors: Todd Fernandez, Lafayette, IN (US); Evan Taylor, Phoenix, AZ (US); Timothy Cale, Phoenix, AZ (US); Brian Neal, Tempe, AZ (US); Jared Stoltzfus, Phoenix, AZ (US)

(21) Appl. No.: 15/323,717

(22) PCT Filed: Jul. 1, 2015
(86) PCT No.: PCT/US15/38903

§ 371 Date: Jan. 3, 2017

Related U.S. Application Data
(60) Provisional application No. 62/020,938, filed on Jul. 3, 2014.

(51) Int. Cl. C12M 1/00; C12M 1/34; C12M 1/33

(52) U.S. Cl. CPC C12M 23/58; C12M 45/02; C12M 29/18; C12M 29/14; C12M 47/10; C12M 41/34; C12M 41/12; C12M 41/26; C12M 41/44

(57) ABSTRACT

A system and method for treating organic material to produce nutrient-rich products are disclosed. Exemplary systems include a first biodigestion reactor and a second biodigestion reactor coupled to the first biodigestion reactor. Exemplary methods include digesting organic material in the first biodigestion reactor and the second biodigestion reactor and recirculating material between the first biodigestion reactor and the second biodigestion reactor.

### CROSS-REFERENCE TO RELATED APPLICATIONS

This application claims the benefit of U.S. Provisional Patent Application Ser. No. 62/020,938, entitled BIODIGESTION REACTOR, SYSTEM INCLUDING THE REACTOR, AND METHODS OF USING SAME, and filed Jul. 3, 2014, the contents of which are hereby incorporated herein by reference to the extent such contents do not conflict with the present disclosure.

### FIELD OF DISCLOSURE

The present disclosure generally relates to biodigestion systems and methods. More particularly, the disclosure relates to biodigestion reactors and systems used to form enriched products and to methods of using the same.

BACKGROUND OF THE DISCLOSURE

Organic material, such as organic waste (e.g., food waste, yard waste, and the like) often ends up in landfills, where it generally adds no value, and potentially produces undesirable products, such as methane gas. To mitigate production of undesirable products in landfills, some communities have instituted procedures for separating organic waste material and composting the organic waste material to form enriched products, such as soil amendments. Although these procedures can work relatively well in some cases, composting requires a relatively large amount of space for the material to be composted, composting is relatively slow and inefficient, composting produces product with relatively low concentrations (typically less than one percent) of desired compounds and thus requires relatively high transportation costs, and composting can result in undesirable odors, particularly when meat or dairy products are being composted.

Accordingly, improved methods and systems for forming enriched products, such as soil amendments, that are more efficient and that produce relatively little odor are desired.

### SUMMARY OF THE DISCLOSURE

Various embodiments of the present disclosure relate to biodigestion systems and methods. While the ways in which various embodiments of the present disclosure address drawbacks of prior biodigestion techniques, in general, various embodiments of the disclosure provide reactors, systems and methods that can be used to convert organic material into enriched products, such as soil amendments, in a relatively time efficient manner.

In accordance with exemplary embodiments of the disclosure, a biodigestion reactor system includes a first reactor comprising a first vessel, a first input for receiving organic material, and one or more microorganisms within the vessel, wherein the one or more microorganisms break down the organic material to form a partially digested composition; a second reactor comprising a second vessel, the second vessel fluidly coupled to the first vessel for receiving the partially digested composition from a first output of the first reactor; a circulation loop coupled to a first output of the second reactor and a second input of the first reactor, and a receiving tank fluidly coupled to a second output of the second reactor. One or more nutrients in a product from the first and/or second reactor can be manipulated by adjusting, for example, one or more of a location of the first output of the second reactor, reaction time, the type and/or concentration of microorganisms in the first and/or second reactor, a temperature or temperature profile (e.g., temporal or spatial) of the first and/or second reactor, a pH in the first and/or second reactor, a number of first and/or second reactors or reactor vessels, an amount of oxygen or oxygenation rate of the first and/or second reactor, and a rate of circulation from the second biodigestion reactor to the first biodigestion reactor or vice versa. The products can be used for, for example, providing nutrients, such as soil amendments and/or fertilizer to a growth medium, such as soil. The organic material can include, for example, organic material selected from one or more of the group consisting of food waste, paper, cardboard, animal waste, and other biodegradable organic material. The one or more microorganisms can include inoculants, such as one or more microorganism types selected from one or more of bacteria and fungi. In accordance with various aspects of these embodiments, the biodigestion reactor system further includes a grinder. In accordance with additional aspects, the biodigestion reactor system includes a hopper to store the organic material. The biodigestion reactor system can also include an active species source to treat the organic material prior to the organic material entering the first reactor, to treat material between reactors (e.g., as a microorganism control), and/or to treat material after treatment from a second reactor. The active species source can comprise an oxidant, such as ozone. The ozone can be formed using, for example, an ozone generator operating at about atmospheric pressure that uses ultraviolet light and/or a corona discharge to form ozone in air. In accordance with further exemplary aspects, one or more of the reactors include a cage and/or mesh to contain solid material particles—e.g., solid particles larger than the openings in the mesh and/or cage. The mesh can be coated with one or more catalysts or cofactors, such as metals (e.g., molybdenum, iron, nickel, magnesium, zinc, or the like). Using a mesh and/or screen can facilitate removal of solid material from the reactor(s), can reduce clogging in a reactor system, and/or can improve pump efficiency in a reactor system. The removed solid material can be discarded, sold as soil amendments, or sent to another reactor for further processing. In accordance with yet further aspects of these embodiments, a system includes multiple first reactors in series and/or in parallel with other first reactors and/or multiple second reactors, in series or in parallel with other second reactors. When in parallel, various first reactors and/or second reactors can be used to, for example, process different types of organic material (e.g., dairy, vegetative, cardboard, or the like). In this case, each reactor could be tuned (e.g., temperatures, pH levels, microorganisms, enzymes, catalysts or cofactors, mesh coatings, and the like) to digest the particular type of organic material. The output from the one or more first reactors and second reactors can be mixed together to form desired final products to meet desired compositions and concentrations of various nutrients. In accordance with further exemplary aspects, an amount and/or type(s) of microorganisms (e.g., pathogens) on a surface of the organic material can be reduced using one or more of: active species treatment, controlling a temperature in one or more of the reactors, and selection of microorganisms in one or more of the reactors.

In accordance with further exemplary embodiments of the disclosure, a method of forming a nutrient-rich composition includes the steps of providing organic material to a first biodigestion reactor; using microorganisms, partially digesting the organic material in a first biodigestion reactor; using a second biodigestion reactor, further digesting the organic material to produce one or more products; and circulating contents from the second reactor to the first biodigestion reactor to tune a composition of the one or more products. In accordance with various aspects of these embodiments, the method further comprises a step of monitoring one or more parameters, such as pH, NH₃, CO₂, temperature, temperature profile (e.g., temporal and/or spatial), NOₓ, humidity, and dissolved oxygen concentration in the first biodigestion reactor and adjusting one or more first and second biodigestion reactor process conditions based on the one or more first biodigestion reactor monitored parameters. In accordance with further aspects, the method comprises a step of monitoring one or more parameters, such as pH, NH₃, CO₂, temperature, NOₓ, humidity, and dissolved oxygen concentration in the second biodigestion reactor and adjusting one or more first and second biodigestion reactor process conditions based on the monitored parameters of the second biodigestion reactor. In accordance with further aspects, the method includes aerobic digestion of the organic material in one or more of the first and second biodigestion reactors. In accordance with yet further aspects, the method includes treating the organic material with an active species, such as an oxidant (e.g., ozone). In accordance with yet further aspects, the nutrient-rich composition comprises one or more of B, Ca, Cu, Fe, Mn, Mg, Mo, N, P, K, Na, Zn, one or more chlorides, one or more sulfates, one or more nitrates, one or more nitrites, one or more carbonates, fulvic acid, and humic acid. In accordance with yet further aspects, the method further includes a step of monitoring a load of a grinder and adjusting one or more process parameters based on the load.

In accordance with additional exemplary embodiments of the disclosure, a biodigestion reactor system includes a grinder to grind organic material; an active species to treat the organic material; a first reactor comprising a first vessel, a first input for receiving the organic material, and one or more microorganisms within the vessel, wherein the one or more microorganisms break down the organic material to form a partially digested composition; a second reactor comprising a second vessel, the second vessel fluidly coupled to the first vessel for receiving the partially digested composition from a first output of the first reactor; a circulation loop coupled to a first output of the second reactor and a second input of the first reactor; a first receiving tank fluidly coupled to a second output of the second reactor; and a second receiving tank fluidly coupled to a third output of the second reactor. In accordance with various aspects of these embodiments, a composition and/or concentration of one or more nutrients in a product from the second reactor is manipulated by adjusting one or more of a location of the first output of the second reactor, reaction time, the type and/or concentration of microorganisms in the first and/or second reactor, a temperature or temperature profile (e.g., spatial and/or temporal) of the first and/or second rector, a pH in the first and/or second reactor, an amount of oxygen or oxygenation rate of the first and/or second reactor, and a rate of circulation from the second biodigestion reactor to the first biodigestion reactor. The organic material can include, for example, organic material from one or more of the group consisting of food waste, paper, cardboard, animal waste, and other biodegradable organic material. The one or more microorganisms can include inoculants, such as one or more microorganism types selected from one or more of bacteria and fungi. In accordance with additional aspects, the biodigestion reactor system includes a hopper to store the organic material. The active species can comprise an oxidant, such as ozone. The ozone can be formed using, for example, an ozone generator operating at about atmospheric pressure that uses ultraviolet light or corona discharge to form ozone in air. The system can include multiple first reactors in series and/or in parallel with each other and/or multiple second reactors in parallel or in series with each other. In accordance with various examples of the disclosure, the first biodigestion reactors can include one or more initial or primary reactors and second biodigestion reactors can include one or more final stage reactors, from which product is drawn. The first and second biodigestion reactors can include various features as described herein.

As set forth in more detail below, systems and methods described herein can be used to treat diverse organic material—e.g., organic material including food waste, such a vegetation, meat, and/or dairy, along with other organic waste. Some exemplary embodiments include dynamic temperature, fluid flow rate (e.g., from other biodigestion reactors), pH, or oxygen flow rates to a biodigestion reactor to accommodate the diverse organic material and/or to account for dynamic digestion conditions as digestion of the organic material changes (e.g., with time and/or with addition of additional organic material).

### BRIEF DESCRIPTION OF THE DRAWING FIGURES

A more complete understanding of exemplary embodiments of the present disclosure can be derived by referring to the detailed description and claims when considered in connection with the following illustrative figures.

FIG. 1 illustrates a system in accordance with exemplary embodiments of the disclosure.

FIG. 2 illustrates a method in accordance with additional exemplary embodiments of the disclosure.

FIG. 3 illustrates an exemplary reactor in accordance with exemplary embodiments of the disclosure.

FIGS. 4(a) and 4(b), 5(a)-5(c), 6 and 7 illustrate exemplary cages and cage assemblies in accordance with additional exemplary embodiments of the disclosure.

FIG. 8 illustrates a multi-reactor system in accordance with yet further exemplary embodiments of the disclosure.

FIG. 9 illustrates a system in accordance with further exemplary embodiments of the disclosure.

It will be appreciated that elements in the figures are illustrated for simplicity and clarity and have not necessarily been drawn to scale. For example, the dimensions of some of the elements in the figures may be exaggerated relative to other elements to help to improve the understanding of illustrated embodiments of the present disclosure.

### DETAILED DESCRIPTION OF EXEMPLARY EMBODIMENTS OF THE DISCLOSURE

The description of exemplary embodiments provided below is merely exemplary and is intended for purposes of illustration only; the following description is not intended to limit the scope of the disclosure or the claims. Moreover, recitation of multiple embodiments having stated features is not intended to exclude other embodiments having additional features or other embodiments incorporating different combinations of the stated features.

As set forth in more detail below, exemplary methods and systems as described herein can be used to convert organic material into nutrient-enriched product(s). The product(s) can be used to supply nutrients, such as soil amendments, to a growth medium, such as soil.

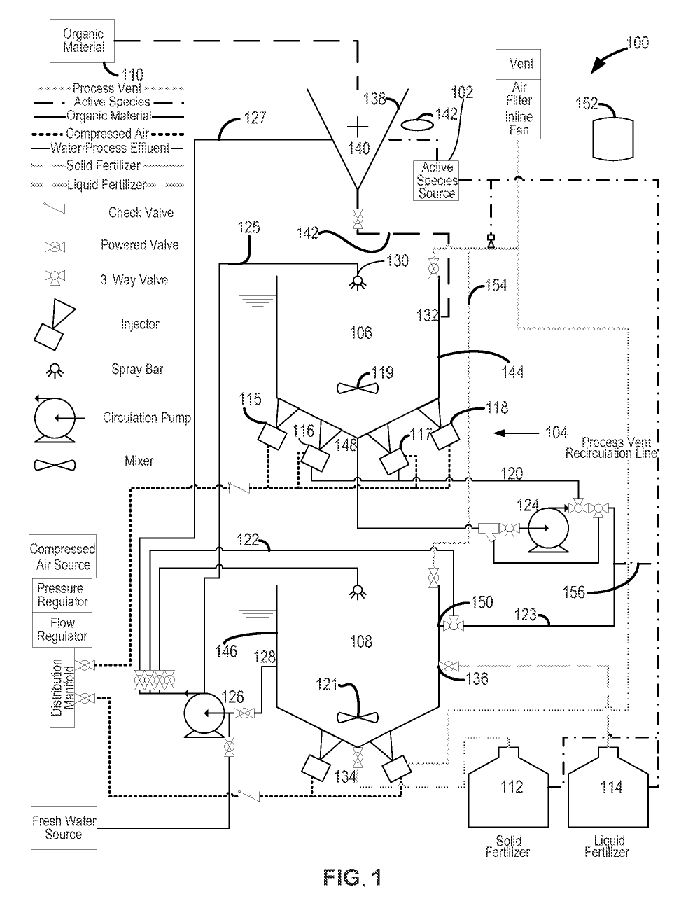

FIG. 1 illustrates an exemplary system 100 in accordance with various embodiments of the disclosure. In the illustrated example, system 100 includes an active species source 102 and a biodigestion reactor system 104. Biodigestion reactor system 104 can include multiple reactors or stages. In the illustrated example, biodigestion reactor system 104 includes a first biodigestion reactor 106 and a second biodigestion reactor 108. Biodigestion reactor systems can include any suitable number of reactors or stages, which may be in series and/or in parallel. For example, exemplary systems can include two or more first reactors 106 that can be in parallel or in series and coupled to one or more second reactors 108. By way of particular examples, multiple first reactors 106 can be coupled together in parallel, and each first reactor 106 can be set up to treat various types of organic material (e.g., dairy, vegetation, cardboard, etc.). The output from each of the first reactors can be combined and fed to one or more second reactors 108. Similarly, exemplary systems can include multiple second reactors 108 coupled together in series or in parallel. By way of examples, system 100 can include 1, 2, 3, 4, 5, 6, 7, 8, 9, 10, or more first and/or second biodigestion reactors, wherein each of the first and/or second biodigestion reactors can be coupled in series and/or in parallel.

During operation of system 100, organic material 110 is converted into nutrient-enriched product(s), such as solid and/or liquid products suitable for use as soil amendments, using biodigestion reactor system 104. The organic material 110 can be treated with active species (e.g., an oxidant, such as ozone) from active species source 102. Treated organic material and/or untreated organic material is then transferred to first biodigestion reactor 106 (e.g., at a first input 132) for treatment, and, in the illustrated example, to second biodigestion reactor 108 for further treatment. Nutrient-enriched products can be collected in vessels 112 (for, e.g., solids) and 114 (for, e.g., liquids).

Organic material 110 can include, for example, one or more materials from the group consisting of food waste, paper, cardboard, animal waste, and other biodegradable organic material. By way of particular examples, organic material 110 includes food waste, which may include meat, dairy, and/or vegetation.

Active species source 102 can include any suitable source of active species. By way of examples, active species source includes an oxidant source. The oxidant source can be an ozone generator. Exemplary ozone generators suitable for use with the present disclosure include corona discharge ozone generators and ultraviolet light ozone generators. Exemplary ozone generators can operate at or near atmospheric pressure. The exemplary generators can produce, for example, greater than 100 ppb ozone in air, greater than 500 ppb ozone in air, greater than 1 ppm ozone in air, or greater than 2 ppm ozone in air. In accordance with various aspects of these examples, ozone can be generated by drawing (e.g., via a pump) air though a reaction chamber of an active species source, and energizing the oxygen atoms—for example, by using ultraviolet light or a corona discharge—to increase a concentration of active species in air. For example, a concentration of ozone can increase from about 10 ppb to greater than 100 ppb ozone in air, greater than 500 ppb ozone in air, greater than 1 ppm ozone in air, or greater than 2 ppm ozone in air. The air with increased active species (e.g., ozone) can then be pumped toward organic material 110 to treat the organic material. The active species (e.g., ozone) can create cellular rupture and lysis—e.g., cellular rupture of e. coli and other microorganisms.

The active species can be used to reduce odors that might otherwise be associated with organic material 110, to break down organic material 110, to increase a surface area to volume ratio of organic material, to kill microorganisms on a surface of organic material 110, to sterilize the organic material, to reduce a number of microorganisms on the surface, to break down toxins (e.g., chlorinated herbicides and/or organochlorine pesticides), break down pharmaceuticals, and/or break down volatile organic compounds in or associated with the organic material. Reducing the number of microorganisms has an added benefit of providing additional process control during processing of organic material 110 in, for example, reactors 106 and 108 of biodigestion system 104. And, the active species can be used to reduce or eliminate pathogenic microorganisms, such as E. coli on a surface of organic material 110.

System 100 can be configured, such that a half-life of the active species is relatively short, such that the active species do not undesirably interfere with downstream processes. For example, the half-life of the active species can be less than 2 hours, less than 1.5 hours, less than 1 hour, about 30 minutes or less, or be about 30 to about 60 minutes.

Biodigestion reactors 106, 108 can include any suitable biodigestion reactor. By way of examples, biodigestion reactor 106 can include a first vessel 144 formed of metal, such as stainless steel or plastic, such as high density polyethylene (HDPE). Biodigestion reactor 108 can similarly include a second vessel 146 formed of metal, such as stainless steel or plastic, such as HDPE. Reactors 106, 108 can include one or more microorganisms, such as one or more inoculants, such as bacteria and/or fungi to break down the organic material into one or more compounds suitable for providing nutrients, such as those noted herein, to a growth medium. Biodigestion in biodigestion reactors 106, 108 can include aerobic digestion of the organic material. For example, the biodigestion in reactors 106, 108 can be greater than 80% aerobic digestion, greater than 90% aerobic digestion, or greater than 95% aerobic digestion. Various microorganisms (an/or oxygen present in the system) can also breakdown toxins and/or pharmaceuticals that are present in the feed organic material.

Exemplary microorganisms suitable for use with the present disclosure and exemplary growth temperatures and pH ranges are provided below in Table 1.

TABLE 1

| | | Exemplary | | |
|---|---|---|---|---|
| | | Temperature Range | | Exemplary |
| Microbe | Exemplary Use | Min C. ° | Max C. ° | pH |
| Azobacter | Atmospheric Nitrogen fixing | 25 | 30 | 7.25 |
| chroococcum | | | | |
| Bacillus | Starch Hydrolysis and | 30 | 40 | 6.3 |
| amyloliquefaciens | Enzymatic Protein Digestion | | | |
| Bacillus azotoformans | Denitrifying | 42 | 46 | 7 |
| Bacillus coagulans | Lactic Acid production | 40 | 50 | 7 |
| Bacillus licheniformis | Controllable for discrete | 37 | 50 | 6.8 |
| | enzyme production at | | | |
| | specific temperature and | | | |
| | pH ranges. Protease | | | |
| | (protein digestion) at high | | | |
| | temperature | | | |
| Bacillus megaterium | Atmospheric Nitrogen fixing | 30 | 42 | 7 |
| Bacillus pumilus | Atmospheric Nitrogen fixing | 37 | 50 | 6.5 |
| Bacillus subtilis | Many beneficial digestion | 25 | 35 | 7 |
| | enzymes such as amylase, | | | |
| | protease, pullulanase, | | | |
| | chitinase, xylanase, lipase, | | | |
| | among others | | | |
| Bacillus thuringiensis | Natural Insecticide as a | 50 | 70 | 7 |
| | result of digestion | | | |
| Paenibacillus durum | Beneficial Soil Microbe for | 40 | 50 | 7 |
| | Crops | | | |
| Paenibacillus | Beneficial Soil Microbe for | 30 | 50 | 8 |
| polymyxa | Crops | | | |
| Pseudomonas | Lipase production | 30 | 35 | 7.2 |
| aureofaciens | | | | |
| Pseudomonas | Kills pathogens, digests | 28 | 30 | 7 |
| fluorescens | lignin | | | |
| Streptomyces | Produces geosmin, a | 25 | 35 | 6.25 |
| griseues | humic acid, and pleasant | | | |
| | “earthy odors” | | | |
| Streptomyces lydicus | Improves yields in some | 30 | 40 | 5.8 |
| | legumes and peas | | | |
| Actinobacteria | Higher Temperature lignin | 40 | 48 | 7 |
| thermomonospora | digestion | | | |
| Actinobacteria | Kills pathogens, digests | 33 | 45 | 7 |
| actinomadura | lignin | | | |
| Actinobacteria | Digests Lignin | 33 | 35 | 7 |
| actinosynnema | | | | |
| Actinobacteria | Digests Lignin and can | 33 | 35 | 7 |
| nocardiopsis | survive in high salt-content | | | |
| | environments (e.g., soy | | | |
| | product waste) | | | |
| Actinobacteria | Breaks down lipids | 33 | 35 | 7 |
| streptoalloteichus | | | | |
| Azospirillum | Colonizes roots to promote | 25 | 30 | 7 |
| Lipoferum | plant growth. | | | |
| Aquaspirillum | Can survive in high salt- | 30 | 32 | 7 |
| magnetotacticum | content environments (e.g., | | | |
| | soy product waste) | | | |
| Cellvibrio mixtus | Cellulose digestion and | 65 | 70 | 7 |
| | high temperature allow for | | | |
| | creative use in primary | | | |
| | reactors to control for | | | |
| | contaminant bacteria or | | | |
| | secondary reactors to | | | |
| | digest the remaining | | | |
| | lignin/cellulose | | | |
| Herbaspirillum | Promotes rice growth, | 25 | 34 | 7 |
| seropedicae | | | | |
| Marinomonas | Can survive in high salt- | 20 | 40 | 7 |
| primoryensis | content environments (e.g., | | | |
| | soy product waste) | | | |
| Acidothermus | Breaks down lignin and | 50 | 60 | 6 |
| cellulolyticus | cellulose into many useful | | | |
| | acids for other bacteria or | | | |
| | plant digestion | | | |
| Agromonas | Nitrogen Fixing at low | 25 | 27 | 7 |
| oligotrophica | temperatures | | | |
| Azomonas agilis | Nitrogen Fixing at low | 20 | 30 | 7.4 |
| | temperatures | | | |
| Azorhizobium | Symbiotic with plant roots | 25 | 30 | 7 |
| caulinodans | and stems | | | |
| Beijerinckia | Breaks down oils | 20 | 30 | 9.5 |
| Bradyrhizobium | Nitrogen fixing and | 25 | 30 | 7 |
| japonicum | symbiotic with plants | | | |
| Derxia gummosa | Symbiotic with plants for | 25 | 35 | 5.5 |
| | tropical soils and nitrogen | | | |
| | fixing | | | |
| Janthinobacterium | Produces anti-fungal | 25 | | 7 |
| lividum | agents for promoting plant | | | |
| | health | | | |
| Rhizobium japonicum | Soybean symbiosis and | 27.4 | 33.7 | 6.6 |
| | nitrogen fixation | | | |
| Sinorhizobium | Reduction of N2 to NH4 | 25 | 30 | 7 |

Biodigestion reactors 106, 108 can also include one or more agitators or mixers to circulate, agitate, homogenize, and/or shear material within the reactors. For example, biodigestion reactor 106 can include one or more venturi injectors 115, 116, 117, 118 to mix material within biodigestion reactor 106. Alternatively, pumps or impellers could be used as the agitators. Use of a venturi injector (e.g., an eductor) may be particularly advantageous, because, in addition to mixing the material, the venturi injector can be used to add air, e.g., at a known or controlled flow rate, to the reactor, which can facilitate aerobic digestion. In addition, using a venturi injector can be advantageous because it can cause material to move and mix that might otherwise be difficult to move or mix with a traditional agitator. Also, use of a venturi injector 115-118 can be used to regulate or assist with regulation of a temperature in a reactor. Biodigestion reactors 106, 108 can additionally or alternatively include other agitators, such as a motor-driven impeller 119, and/or other aerators.

As noted above, first biodigestion reactor 106 and/or second biodigestion reactor 108 can be configured to accommodate one or more types of organic material (e.g., food waste and/or particular types of food waste, such as vegetation, dairy, meat, etc., paper, cardboard, animal waste, and other biodegradable organic material). For example, system 100 can include one first biodigestion reactor 106 that is configured to digest a plurality of types of organic material. Or, system 100 can include two or more first biodigestion reactors, wherein one or more of the first biodigestion reactors are configured to digest particular type(s) of organic material. For example, the first biodigestion reactors can run at particular temperatures, pH levels, and/or include particular microorganisms that favor the breakdown of the organic material feed into one or more nutrient-rich products. The output from the plurality of first biodigestion reactors 106 can be fed to one or more second biodigestion reactors 108. Each biodigestion reactor 106, 108 can be sized to accommodate an amount material to be processed.

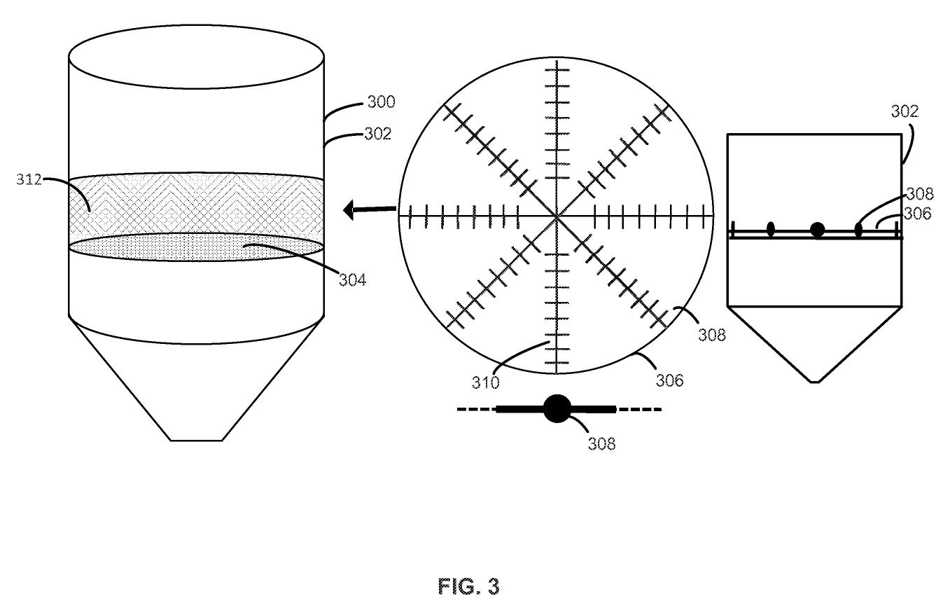

One or more of biodigestion reactors 106, 108 can include a filter between a reactor input (e.g., input 132) and an output (e.g., first output 148 of first reactor 106). Additionally or alternatively, system 100 can include filters between input 150 and second output 134 and/or third output 136 of second biodigestion reactor 108. An exemplary filter can include a mesh, having opening with an average cross sectional size of about 3 mm to about 4 mm. The filter can advantageously be removable to allow for cleaning and maintenance. Additionally or alternatively, one or more of first biodigestion reactor 106 and second biodigestion reactor 108 can include a screen (or mesh) and/or a cage, as described in more detail below in connection with FIGS. 3-8. Additionally or alternatively, biodigestion reactors can include one or more membranes that retain bacteria and/or enzymes, and allow nutrient-rich product to pass through the membrane. Such membranes may be particularly useful in a second biodigestion reactor.

Biodigestion reactors 106, 108 can be configured for different types of reactions. For example, first biodigestion reactor 106 can be configured to provide relatively high microorganism growth rate. By way of examples, first biodigestion reactor 106 can operate at a temperature of about 25° C. to about 72° C. or about 60° C. to about 72° C. or about 25° C. to about 55° C. or about 30° C. to about 40° C. The pH of first biodigestion reactor can range from about 4.8-9 or about 5-8. The temperature can also be controlled to facilitate or discourage growth of certain microorganisms; the microorganisms can be tuned to digest certain types of material. For example, the temperature can be set at a temperature high enough to kill undesired microorganisms, such as pathogens, such as E. coli, and/or encourage growth of other microorganisms, including microorganisms that kill certain pathogens, such as E. coli and/or that can digest certain types of organic material, such as cellulose-based material. In these cases, for example, the biodigestion reactor can be run at a temperature of about 60° C. to about 72° C. to tune the microorganisms (e.g., kill unwanted pathogens) and/or to encourage growth of certain microorganisms, such as those that breakdown cellulose.

Second biodigestion reactor 108 can be configured to provide relatively high enzyme production from a reaction of the microorganisms and the organic material. By way of examples, second biodigestion reactor 108 can operate at a temperature of about 25° C. to about 72° C. or about 25° C. to about 55° C. or about 30° C. to about 40° C. or about 60° C. to about 72° C.—e.g., for the same reasons noted above. The pH of first biodigestion reactor can range from about 4.8-9 or about 5-8.

In accordance with some exemplary embodiments of the disclosure, one or more of a temperature, pH, and oxygen supply rate to first biodigestion reactor 106 and/or second biodigestion reactor 108 can be varied to control selectivity of microorganisms within the respective reactor. Further, a temperature of first biodigestion reactor 106 and/or second biodigestion reactor 108 can be manipulated during processing to, for example, initially favor higher digestion rates (e.g., at a higher temperature) and then to control selected microorganism growth and/or increased enzyme production (e.g., at a lower temperature). Additionally or alternatively, a temperature can be manipulated to increase acid (e.g., humic acid or fulvic acid) production.

System 100 also includes one or more circulation lines 120, 122, 123, 125, 127 which can include one or more circulation pumps 124, 126. Material from second biodigestion reactor 108 (e.g., received from a first output 128 of second biodigestion reactor 108) can be provided to first biodigestion reactor 106 (e.g., a second input 130) using line 125 and pump 126. Similarly, material from second biodigestion reactor 108 can be provided to first biodigestion reactor 106 using line 120 and pump 124. Material circulated from second biodigestion reactor 108 to first biodigestion reactor 106 can be used to control reactions and reaction rates in both first biodigestion reactor 106 and second biodigestion reactor 108. Controlling the reactions in the respective biodigestion reactors can, in turn, allow control of products and nutrient concentrations from biodigestion reactors system 104. Furthermore, a location of output 136 and/or input 150 can be used to control desired and/or undesired reactions within the respective biodigestion reactors. For example, an output 136 may be raised or lowered depending on desired material to be circulated to first biodigestion reactor 106. Similarly, input 132 and/or 130 can be moved to “feed” one or more regions within first biodigestion reactor 106. System 100 can also include automated or manual back flush systems on one or more of the lines to prevent, mitigate, or reverse clogging in various lines of the system to or from reactors 106, 108. In the illustrate example, line 127 can be used to provide liquid from second biodigestion reactor 108 to grinder 140 to reduce an amount of water that might otherwise be added to system 100 to facilitate grinding of organic material 110. The circulated material in line 127 can also facilitate breakdown of organic material. Although not illustrated, material from first biodigestion reactor 106 can similarly be used to treat organic material 110. Further, exemplary systems can use feedback from one or more of circulation pumps 124, 126 to manipulate one or more process parameters (e.g., dilution of material within the reactor (e.g., dilute material in reactor 106 with material from reactor 108), change pump speed, or the like) of first biodigestion reactor 106 and/or second biodigestion reactor 108.

System 100 can also include gas circulation lines 154 to allow for gas produced from one reactor to be introduced into another reactor. For example, NH₃, CO₂, NOₓ, or the like can be fed from biodigestion reactor 106 to biodigestion reactor 108 or vice versa or to a final product in vessel 112 or 114. Additionally or alternatively, gas output from a first biodigestion reactor can be fed to another first biodigestion reactor and/or from a second biodigestion reactor to another second biodigestion reactor. Feeding gasses from one reactor to another can be used to control nutrient content, a pH within a reactor, and/or promote or inhibit growth of particular microorganisms, and/or promote digestion of organic material.

As noted above, nutrient-enriched products can be collected in vessels 112, 114. For example, (e.g., solid) products from a second output 134 of second biodigestion reactor 108 can be collected in vessel 112. And, (e.g., liquid) products can be collected in vessel 114 from a third output 136 of second biodigestion reactor 108. A composition and/or concentration of the products can be based on a location of the second and/or third outputs. The active species source 102 can be used to treat one or more products in vessels 112, 114 and/or material between biodigestion reactors in, for example, line 156. For example, if it is desired to stop or mitigate growth of one or more microorganisms in the product(s), the active species source can be used to reduce a number of microorganisms in the product(s). Additionally or alternatively, the product(s) can be subjected to a pasteurization process.

The liquid and solid products can include nutrients that can be used as soil amendments. Various nutrients include biologically available nutrients, such as one or more of B, Ca, Cu, Fe, Mn, Mg, Mo, N, P, K, Na, Zn, one or more chlorides, one or more sulfates, one or more nitrates, one or more nitrites, one or more carbonates, fulvic acid, and humic acid.

System 100 can also include a hopper 138 to hold organic material. System 100 can also include a grinder 140 to cut organic material 110 into smaller pieces, (e.g., pieces having a largest dimension of about 2000 microns for pre-processing then down to 300-600 microns for typical agricultural markets, 100-200 microns for retail markets, and 10-50 microns for hydroponics/aeroponics markets). Use of grinder 140 can increase a surface area of organic material available for reaction in biodigestion system 104.

System 100 can also include an evaporator (not illustrated) coupled to one or more outputs of a biodigestion reactor, such as second biodigestion reactor 108.

Exemplary systems, such as system 100, can also include one or more sensors. For example, system 100 can include an active species sensor 142. Active species sensor 142 can be located anywhere between active species source 102 and first input 132.

System 100 can include one or more of an NH₃ sensor, a dissolved oxygen sensor, a pH sensor, a CO₂ sensor, a temperature sensor, an NOₓ sensor, a humidity sensor, and a pressure sensor coupled to one or more of the first biodigestion reactor 106 and the second biodigestion reactor 108. The sensors can be used to monitor reactions within the respective biodigestion reactors and one or more process parameters, such as mixing rate, circulation rate, amount of microorganisms, types and species of microorganisms, and the like, and can be automatically or manually manipulated based on sensor values. Furthermore, one or more sensors and/or sensor types can be located at various locations (e.g., heights) of the first biodigestion reactor vessel 144 and/or second biodigestion reactor vessel 146 to monitor various reactions at the respective locations of the biodigestion reactors.

System 100 can also include one or more breeder reactors 152. The breeder reactors can be used to incubate or grow one or more microorganisms for use in one or more of first biodigestion reactor 106 and second biodigestion reactor 108. Breeder reactor 152 can include suitable nutrient, water, active species (e.g., ozone), and/or air supplies.

FIGS. 3-8 illustrate exemplary reactor cage designs and mesh designs in accordance with further exemplary embodiments of the disclosure. The cage and mesh designs describe below can allow for smaller pieces of organic material to pass through the screen/mesh, while retaining larger pieces for further digestion and/or removal from a biodigestion reactor.

FIG. 3 illustrates a tank 300, which can be used as a first biodigestion reactor or a second biodigestion reactor, and may be particularly well suited for use as a biodigestion reactor that includes solid material, such as a first biodigestion reactor. In the illustrated example, reactor 300 includes a vessel 302 and a screen 304, and optional wall 312 to retain solid material during removal. A mesh size of screen 304 can vary according to a number of factors, including type(s) of organic material to be treated, a number of first reactor stages in series, and the like. By way of examples, the mesh size can have an opening cross section of about 3 mm to about 10 mm or about 4 mm to about 5 mm. Reactor 300 can also include a rotating disc 306, which can include cutting or grinding devices 308 to facilitate cutting and/or grinding of organic material within reactor 300. One or more cutting/grinding devices 308 can be coupled (e.g., rotatably coupled) to a bar 310. Screen 304, rotating disc 306, cutting devices 308, and bars 310 can be formed of any suitable materials, including stainless steel and plastics. Screen 304 and/or wall 312 may be coated with catalysts and/or cofactors as described herein

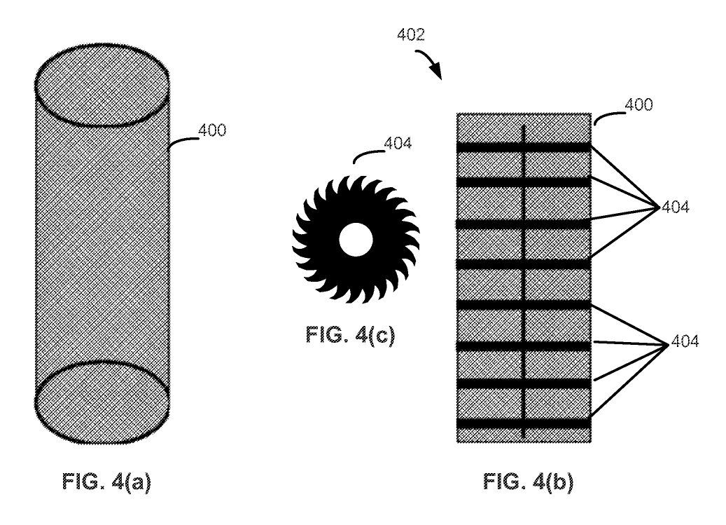

FIG. 4(a) illustrates a cage 400, which is suitable for, for example, retaining organic material during processing in a biodigestion reactor and/or transporting organic material between biodigestion reactors. Cage 400 can be formed of mesh material, such as a screen having an opening cross sectional size of about 3 mm to about 10 mm or about 4 mm to about 5 mm. FIG. 4(b) illustrates a cage assembly 402, which includes cage 400 and one or more cutting devices 404, illustrated in FIG. 4(c). Cutting devices 404 can be configured as, for example, straight or curved blades and can be used to cut or grind organic material within a biodigestion reactor, such as a first biodigestion reactor or a second biodigestion reactor. In the illustrated example, cage 400 and cage assembly 402 are cylindrical.

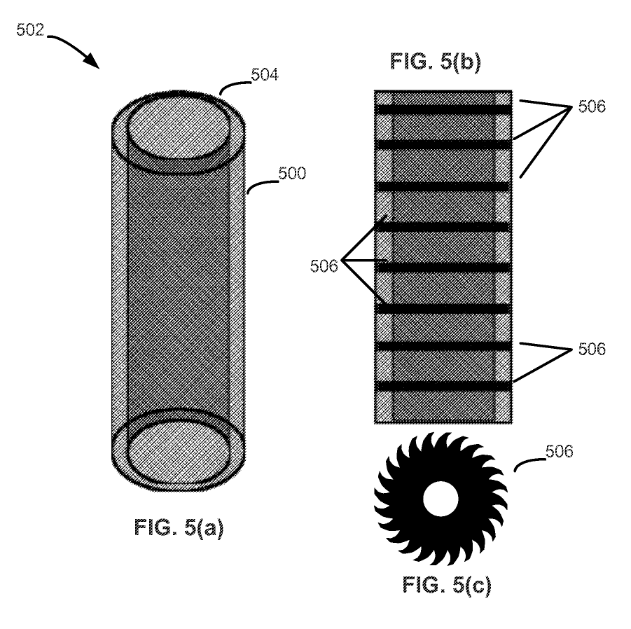

FIGS. 5(a)-5(c) illustrate another cage assembly 502 that includes a first cage 500 and a second cage 504. In the illustrated example, cage assembly 502 is cylindrical. The mesh size of the cages 500, 504 can be the same or similar to other mesh sizes noted herein. Organic material can be retained between first cage 500 and second cage 504 during processing in a biodigestion reactor, such as the first and second biodigestion reactors described herein. Cage assembly 502 can also include cutting devices 506 to facilitate cutting or grinding of organic material within a biodigestion reactor.

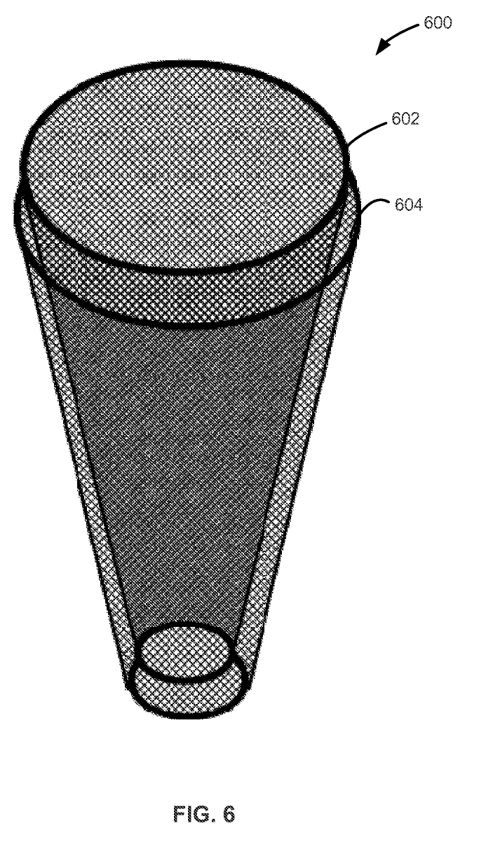

FIG. 6 illustrates another exemplary cage assembly 600, including a first cage 602 and a second cage 604. In the illustrated example, cage 602 and cage 604 are frusto-conical shaped; the mesh sizes of the screens can be the same or similar to other mesh sizes noted herein. Cage assembly 600 can be used in connection with any biodigestion reactor, including the biodigestion reactors disclosed herein. During use, organic material can be retained between first cage 602 and second cage 604. The cages can be moved relative to each other (e.g., cage 604 can be moved downward relative to cage 602) to cause grinding or cutting of organic material retained between first cage 602 and second cage 604.

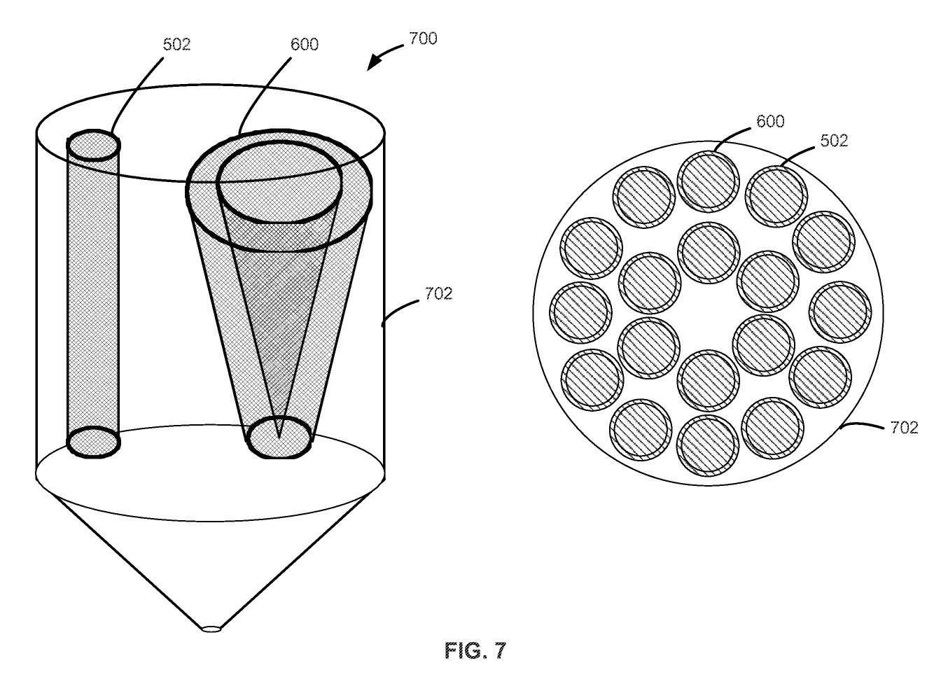

FIG. 7 illustrates a biodigestion reactor 700, including a vessel 702 and one or more cage assemblies 600, 502. A biodigestion reactor can include any suitable number and/or types of cages or cage assemblies. Each cage or cage assembly can include the same or different types of organic material. For example, one cage assembly could be used for vegetation and another cage assembly could be used for meat. The grinding devices of the cage assemblies can vary according to application—e.g., meat, vegetation, cardboard, and the like.

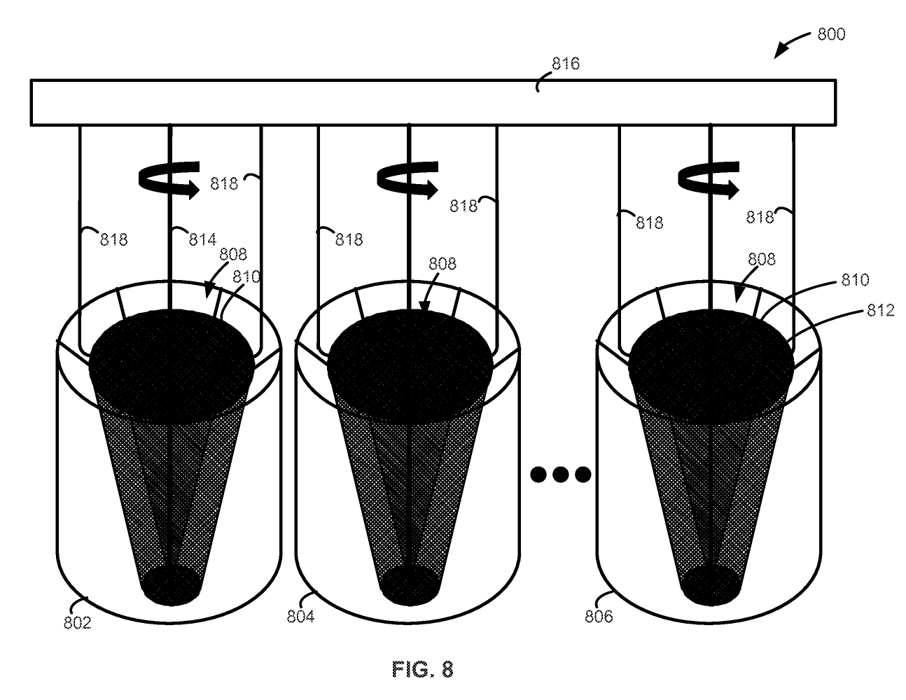

FIG. 8 illustrates a multi-reactor system 800 in accordance with further exemplary embodiments of the disclosure. Multi-reactor system 800 includes a first biodigestion reactor 802, a second biodigestion reactor 804, and a third biodigestion reactor 806, which can be in series and/or in parallel with each other. Although illustrated with three biodigestion reactors, system 800 can includes any suitable number of biodigestion reactors. In the illustrated example, each biodigestion reactor 802-806 includes a cage assembly 808, which can be the same or similar to any of the cage assemblies described above. By way of illustrative example, cage assembly 808 includes a first cage 810 to retain organic material and a second cage 812 to facilitate cutting or grinding of the organic material. Second case 812 can rotate about an axis 814. Further, the plurality of cage assemblies can be coupled together—e.g., via rail 816 and retaining devices 818, such that cage assemblies 808 can be moved between biodigestion reactors (e.g., one or more first and/or second biodigestion reactors) using rail 816. Alternatively, rail 816 can be used to remove cage assemblies 808 from their respective biodigestion reactors. Further, multi-reactor system 800 can be configured to transfer liquid from one or more other reactors, which can be in series or parallel to each other.

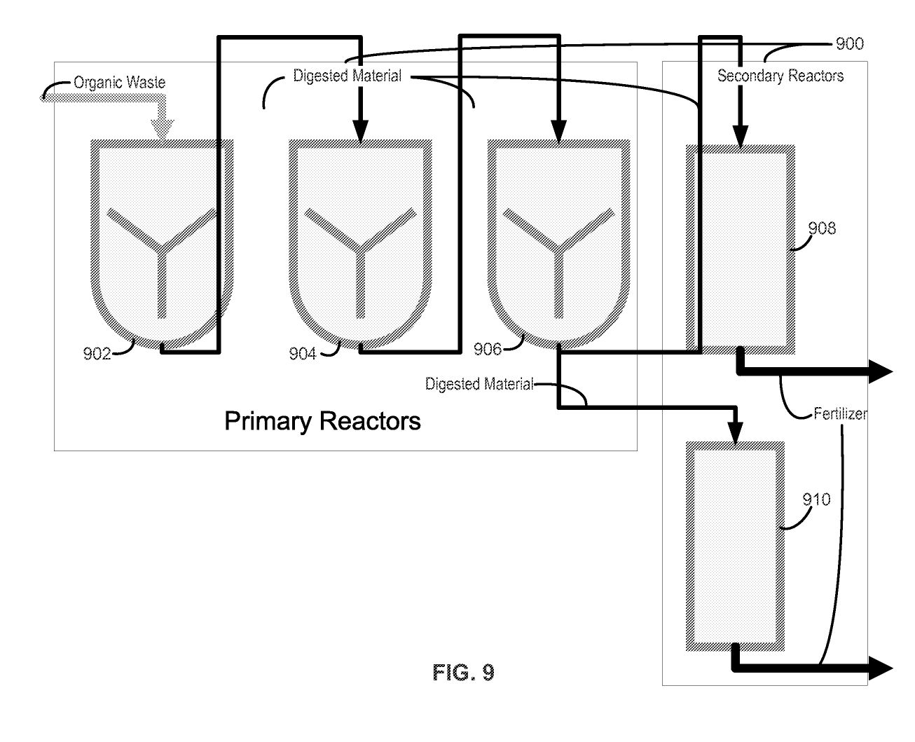

As noted above, exemplary biodigestion reactor systems can include multiple reactors or stages. FIG. 9 illustrates a system 900 that includes multiple first reactors 902-906 coupled together in series and multiple second reactors 908, 910 coupled together in parallel. Systems 900 can include any of the components described above, such as pumps, active species source, breeder reactor and the like. Although illustrated with three first biodigestion reactors 902-906 in series and two second biodigestion reactors 908-910 in parallel, systems in accordance with the present disclosure can include and suitable number of first biodigestion reactors in series and/or in parallel and second biodigestion reactors in series and/or in parallel.

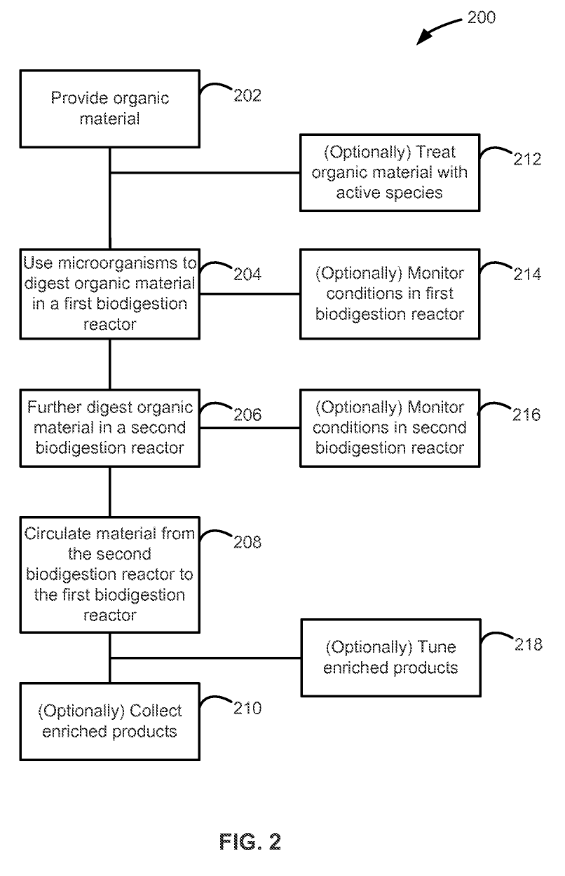

Turning now to FIG. 2, a method 200 of forming a nutrient-rich composition is illustrated. Method 200 includes the steps of providing organic material (step 202), using microorganisms, partially digesting the organic material in a first biodigestion reactor (step 204), using a second biodigestion reactor, further digesting the organic material to produce one or more products (step 206), and circulating contents from the second biodigestion reactor to the first biodigestion reactor (step 208). As illustrated, method 200 can also optionally include additional steps, such as one or more of collecting enriched products (step 210), treating organic material with active species (step 212), monitoring reactor conditions of the first biodigestion reactor (step 214), monitoring reactor conditions of the second biodigestion reactor (step 216), and tuning the composition and/or concentration of the enriched products (step 218).

During step 202, organic material is provided. Exemplary materials suitable for step 202 include organic material noted above in connection with organic material 110.

During step 212, the organic material can optionally be treated with active species, such as the active species noted above. For example, the organic material can be treated with an active species, such as an oxidant (e.g., ozone) prior to entering a first biodigestion reactor. In the case of ozone, the ozone can be created using a corona discharge ozone source and/or an ultraviolet light ozone source. Step 212 can include treating the organic material for about 30 min to about 3 hr, or about 15 min to about 24 hr. Further, step 212 can include allowing about 100 ppm to about 300 ppm or about 10 ppm to about 3000 ppm of ozone to pass over or through the organic mixture.

As noted above, the active species may be desirably allowed to dissipate and/or decay or degrade prior to entering a first biodigestion reactor. In some cases, the active species is selected, such that it decays and/or dissipates prior to material entering a biodigestion reactor or another stage. This allows treatment of the organic material with the active species and before entering a first biodigestion reactor, without interfering with subsequent method steps.

During step 204, organic material (which can include organic material treated with active species and/or organic material that has not been treated with active species) is partially digested in a first biodigestion reactor (e.g., biodigestion reactor 106) using microorganisms. The microorganisms can be the same as those described above and can include one or more types of microorganisms, such as one or more fungi and/or one or more bacteria. The microorganisms can be selected such that the digestion of the organic material comprises aerobic digestion. For example, greater than 80%, greater than 90%, or greater than 95% of the digestion can be aerobic. Step 204 can be configured for relatively high rates of microorganism grown.

A temperature of the first biodigestion reactor can range from about 25° C. to about 72° C. or about 25° C. to about 55° C. or about 30° C. to about 40° C. or about 60° C. or about 72° C. (e.g., to kill undesired pathogens and/or to encourage digestion of organic material, such as cellulose). The pH of the first biodigestion reactor can range from about 4.8-9 or about 5-8.

One or more agitators, such as impellers or venturi injectors, can be used to circulate or mix material during step 204. Further, step 204 can include spraying organic material—e.g., from the first biodigestion reactor and/or the second biodigestion reactor—onto a surface of the organic material in the second biodigestion reactor.

Optional step 214 includes monitoring one or more first biodigestion reactor conditions. By way of examples, one or more of NH₃ content, CO₂ content, dissolved oxygen concentration, temperature, NOₓ, humidity, and pH can be monitored during step 214. Furthermore, one or more process conditions can be manipulated based on the monitored conditions. For example, a circulation rate and/or location can be manipulated, a temperature can be adjusted, organic material feed can be adjusted, an amount and/or type of one or more microorganisms can be adjusted, and the like.

At step 206, the organic material is further digested in a second biodigestion reactor using microorganisms. The microorganisms can be the same or different from and in the same or different concentrations as the microorganisms used in the first biodigestion reactor.

A temperature of the second biodigestion reactor can range from about 25° C. to about 72° C. or about 25 to about 55° C. or about 30° C. to about 40° C. The pH of the second biodigestion reactor can range from about 4.8-9 or about 5-8.

One or more agitators, such as impellers or venturi injectors, can be used to circulate or mix material during step 206. Additionally or alternatively, a spray bar can be used to mix the material. Further, step 206 can include spraying organic material—e.g., from the first biodigestion reactor and/or the second biodigestion reactor—onto a surface of the organic material in the second biodigestion reactor.

Optional step 216 includes monitoring one or more second biodigestion reactor conditions. By way of examples, one or more of NH₃ content, CO₂ content, dissolved oxygen concentration, temperature, and pH can be monitored during step 216. Furthermore, one or more process conditions can be manipulated based on the monitored conditions. For example, a circulation rate and/or location can be manipulated, a temperature can be adjusted, organic material feed can be adjusted, a humidity can be adjusted, an amount of NO can be adjusted, an amount and/or type of one or more microorganisms can be adjusted, and the like.

During step 208, material is circulated from the second biodigestion reactor to the first biodigestion reactor. The circulation can be used to provide desired concentrations and/or types of microorganisms from the second biodigestion reactor to the first biodigestion reactor, to provide partially digested organic material from the second biodigestion reactor to the first biodigestion reactor, to manipulate one or more process conditions of the first and/or second biodigestion reactors, and the like.

Finally, during step 210, one or more enriched products can be collected. The enriched products can include solids and/or liquids. Exemplary products can contain, for example, one or more of B, Ca, Cu, Fe, Mn, Mg, Mo, N, P, K, Na, Zn, one or more chlorides, one or more sulfates, one or more nitrates, one or more nitrites, one or more carbonates, fulvic acid, and humic acid.

During step 218, various process conditions of method 200 can be manipulated to tune a composition of the products and/or concentrations of various components, such as one or more of B, Ca, Cu, Fe, Mn, Mg, Mo, N, P, K, Na, Zn, one or more chlorides, one or more sulfates, one or more nitrates, one or more nitrites, one or more carbonates, fulvic acid, and humic acid. For example, a circulation rate, a location of an output of the second biodigestion reactor and/or an input of the first biodigestion reactor in a circulation line can be manipulated, and a location of where products are drawn from the second biodigestion reactor can be manipulated. One or more of a temperature, pH, agitation rate, oxygen feed, or the like can also be manipulated to tune the composition.

Although not illustrated, methods in accordance with exemplary embodiments of the disclosure can include controlling a speed of a grinder. The grinder speed can be manipulated based on, for example, types of organic material and/or desired sizes of pieces of the organic material and/or desired surface area-to-volume ratio of the organic material. Further, an electrical load on a grinder can be monitored. The grinder electrical load can be indicative of types of organic material and/or amount of organic material. One or more process parameters can be adjusted based on the electrical load of the grinder.

The subject matter of the present disclosure includes all novel and nonobvious combinations and sub combinations of the various systems, components, and configurations, and other features, functions, acts, and/or properties disclosed herein, as well as any and all equivalents thereof.

Various examples of the disclosure include
1. A biodigestion reactor system comprising:

a first reactor comprising a first vessel, a first input for receiving organic material, and one or more microorganisms within the vessel, wherein the one or more microorganisms breakdown the organic material to form a partially digested composition;

a second reactor comprising a second vessel, the second vessel fluidly coupled to the first vessel for receiving the partially digested composition from a first output of the first reactor;

a circulation loop coupled to a first output of the second reactor and a second input of the first reactor; and

a receiving tank fluidly coupled to a second output of the second reactor,

wherein a concentration of one or more products from the second reactor is manipulated by adjusting one or more of a location of the first output of the second reactor, reaction time, the microorganisms, a number of first reactors, a number of second reactors, and a rate of circulation.
2. The biodigestion reactor system of example 1, further comprising a grinder and hopper coupled to the first input.
3. The biodigestion reactor system of any of examples 1-2, further comprising an active species source to treat the organic material prior to the organic material entering the first reactor.
4. The biodigestion reactor system of any of examples 1-3, wherein the organic material comprises material selected from one or more of the group consisting of food waste, paper, cardboard, animal waste, and other biodegradable organic material.
5. The biodigestion reactor system of any of examples 1-4, wherein the first reactor comprises one or more first reactor agitators.
6. The biodigestion reactor system of any of examples 1-5, wherein the one or more microorganisms are selected from one or more of bacteria and fungi.
7. The biodigestion reactor system of any of examples 1-6, wherein the second reactor comprises one or more second reactor agitators.
8. The biodigestion reactor system of any of examples 1-7, further comprising a back flush system for the first reactor.
9. The biodigestion reactor system of example 8, wherein the back flush system for the first reactor is fluidly coupled to the second reactor.
10. The biodigestion reactor system of any of examples 5-9, wherein the one or more first reactor agitators comprise a pump.
11. The biodigestion reactor system of any of examples 1-10, wherein the one or more products contain one or more soil amendments.
12. The biodigestion reactor system of any of examples 1-11, wherein the one or more products contain one or more of B, Ca, Cu, Fe, Mn, Mg, Mo, N, P, K, Na, Zn, one or more chlorides, one or more sulfates, one or more nitrates, one or more nitrites, one or more carbonates, fulvic acid, and humic acid.
13. The biodigestion reactor system of any of examples 1-12, wherein a pH fluid in the second vessel ranges from about 4.8 to about 9.
14. The biodigestion reactor system of any of examples 1-13, wherein the second reactor comprises a plurality of outlets and one or more products are extracted from one or more of the plurality of outlets based on a desired composition of the one or more products.
15. The biodigestion reactor system of any of examples 1-14, further comprising an evaporator coupled to an output of the second reactor.
16. The biodigestion reactor system of any of examples 1-15, further comprising an active species sensor upstream of the first input for receiving organic material.
17. The biodigestion reactor system of any of examples 1-16, further comprising one or more sensors selected from the group consisting of NH₃, CO₂, temperature, pH, an NOₓ, a humidity, and a dissolved oxygen concentration coupled to the first reactor.
18. The biodigestion reactor system of any of examples 1-17, further comprising one or more sensors selected from the group consisting of NH₃, CO₂, temperature, pH, an NOₓ, a humidity, and a dissolved oxygen concentration coupled to the second reactor.
19. The biodigestion reactor system of any of examples 1-18, wherein biodigestion in the first reactor comprises aerobic digestion.
20. The biodigestion reactor system of any of examples 1-19, wherein biodigestion in the first reactor comprises greater than 80% aerobic digestion.
21. The biodigestion reactor system of any of examples 1-20, wherein biodigestion in the first reactor comprises greater than 90% aerobic digestion.
22. The biodigestion reactor system of any of examples 1-21, wherein biodigestion in the first reactor comprises greater than 95% aerobic digestion.
23. The biodigestion reactor system of any of examples 1-22, wherein biodigestion in the second reactor comprises aerobic digestion.
24. The biodigestion reactor system of any of examples 1-23, wherein the first reactor comprises a plurality of sensors in the first vessel to monitor a composition of the partially digested composition at a plurality of heights within the first vessel.
25. The biodigestion reactor system of any of examples 1-24, wherein the second reactor comprises a plurality of sensors in the second vessel to monitor a composition of the one or more products at a plurality of heights within the vessel.
26. The biodigestion reactor system of any of examples 1-25, wherein the first reactor comprises a filter between the first input and the output of the first reactor.
27. The biodigestion reactor system of any of examples 1-26, wherein one or more of the first agitator and the second agitator comprise a venturi injector.
28. The biodigestion reactor system of any of examples 1-27, wherein process conditions in the first reactor are configured for aerobic microorganism growth.
29. The biodigestion reactor system of any of examples 1-28, wherein process conditions in the second reactor are configured for enzyme production.
30. The biodigestion reactor system of any of examples 1-29, further comprising one or more additional biodigestion reactors fluidly coupled to one or more of the first reactor and the second reactor.
31. The biodigestion reactor system of any of examples 1-30, further comprising one or more breeder reactors.
32. A method of forming a nutrient-rich composition, the method comprising the steps of:

providing organic material;

using microorganisms, partially digesting the organic material in a first biodigestion reactor;

using a second biodigestion reactor, further digesting the partially digested organic material to produce one or more products; and

circulating contents from the biodigestion second reactor to the first biodigestion reactor to tune a composition of the one or more products.
33. The method of example 32, further comprising a step of monitoring a pH of partially digested organic material in the first biodigestion reactor and adjusting one or more first biodigestion reactor process conditions based on the pH of the partially digested organic material.
34. The method of any of examples 32-33, further comprising a step of monitoring an amount of NH3 from the first biodigestion reactor and adjusting one or more first biodigestion reactor process conditions based on the amount of NH3.
35. The method of any of examples 32-34, further comprising a step of monitoring an amount of CO2 from the first biodigestion reactor and adjusting one or more first biodigestion reactor process conditions based on the amount of one or more of CO2, NOx, and humidity.
36. The method of any of examples 32-35, further comprising a step of monitoring a pH of contents in the first biodigestion reactor and adjusting one or more first biodigestion reactor process conditions based on the pH of the contents in the second biodigestion reactor.
37. The method of any of examples 32-36, further comprising a step of monitoring dissolved oxygen concentration in the first biodigestion reactor and adjusting one or more first biodigestion reactor process conditions based on the dissolved oxygen concentration in the second biodigestion reactor.
38. The method of any of examples 32-37, further comprising a step of monitoring temperature in the first biodigestion reactor and adjusting one or more first biodigestion reactor process conditions based on temperature in the second biodigestion reactor.
39. The method of any of examples 32-38, further comprising a step of monitoring an amount of NH3 from the second biodigestion reactor and adjusting one or more second biodigestion reactor process conditions based on the amount of NH3 from the second biodigestion reactor.
40. The method of any of examples 32-39, further comprising a step of monitoring an amount of CO2 from the second biodigestion reactor and adjusting one or more second biodigestion reactor process conditions based on one or more of the amount of CO2, NOx, and humidity from the second biodigestion reactor.
41. The method of any of examples 32-40, further comprising a step of monitoring a pH of contents in the second biodigestion reactor and adjusting one or more second biodigestion reactor process conditions based on the pH of the contents in the second biodigestion reactor.
42. The method of any of examples 32-41, further comprising a step of monitoring dissolved oxygen concentration in the second biodigestion reactor and adjusting one or more second biodigestion reactor process conditions based on the dissolved oxygen concentration in the second biodigestion reactor.
43. The method of any of examples 32-42, further comprising a step of monitoring temperature in the second biodigestion reactor and adjusting one or more second biodigestion reactor process conditions based on temperature in the second biodigestion reactor.
44. The method of any of examples 32-43, further comprising a step of treating the organic material with an active species.
45. The method of example 44, wherein the active species comprises ozone.
46. The method of any of examples 44-45, further comprising a step of adjusting an amount of active species treatment based on a composition of the organic material.
47. The method of any of examples 32-46, wherein the one or more products contain one or more soil amendments.
48. The method of any of examples 32-47, wherein the one or more products contain one or more of B, Ca, Cu, Fe, Mn, Mg, Mo, N, P, K, Na, Zn, one or more chlorides, one or more sulfates, one or more nitrates, one or more nitrites, one or more carbonates, fulvic acid, and humic acid.
49. The method of any of examples 32-48, further comprising a step of monitoring a load of a grinder and adjusting one or more process parameters based on the load.
50. The method of any of examples 32-49, further comprising a step of controlling a surface-to-volume ratio of the organic material by controlling a speed of the grinder.
51. The method of any of examples 32-50, wherein, during the step of using microorganisms, partially digesting the organic material in a first biodigestion reactor, an agitator is used to mix the partially digested organic material.
52. The method of any of examples 32-51, wherein, during the step of using the microorganisms, partially digesting the organic material in a biodigestion reactor, a venturi injector is used to mix partially digested organic material.
53. The method of any of examples 32-52, wherein, during the step of using the second biodigestion reactor, an agitator is used to mix the organic material.
54. The method of any of examples 32-53, wherein, during the step of using the second biodigestion reactor, a spray bar is used to mix the organic material.
55. The method of any of examples 32-54, wherein the step of using the microorganisms, partially digesting the organic material in a first biodigestion reactor is at a temperature of about 25° C. to about 55° C. or about 60° C. to about 72° C.
56. The method of any of examples 32-55, further comprising a step of spraying organic material onto a surface of the organic material in the second biodigestion reactor.
57. The method of any of examples 32-56, further comprising a step of allowing the organic material in the second biodigestion reactor to settle prior to removing one or more products from the second biodigestion reactor.
58. A biodigestion reactor system comprising:

a grinder to grind organic material;

an active species to treat the organic material;

a first reactor comprising a first vessel, a first input for receiving the organic material, and one or more microorganisms within the vessel, wherein the one or more microorganisms break down the organic material to form a partially digested composition;

a second reactor comprising a second vessel, the second vessel fluidly coupled to the first vessel for receiving the partially digested composition from a first output of the first reactor;

a circulation loop coupled to a first output of the second reactor and a second input of the first reactor;

a first receiving tank fluidly coupled to a second output of the second reactor; and

a second receiving tank fluidly coupled to a third output of the second reactor.
59. The biodigestion reactor system of example 58, wherein a concentration of one or more products from the second reactor is manipulated by adjusting one or more of a location of the first output, time, the microorganisms, concentration of the microorganisms, and a rate of circulation.
60. The biodigestion reactor system of any of examples 58-59, wherein the organic material comprises material selected from one or more of the group consisting of food waste, paper, cardboard, animal waste, and other biodegradable organic material.
61. The biodigestion reactor system of any of examples 58-60, wherein the first reactor comprises one or more first reactor agitators.
62. The biodigestion reactor system of any of examples 58-61, wherein the one or more microorganisms comprise one or more of bacteria and fungi.
63. The biodigestion reactor system of any of examples 58-62, wherein the second reactor comprises a second reactor agitator.
64. The biodigestion reactor system of any of examples 58-63, further comprising a back flush system for the first reactor.
65. The biodigestion reactor system of example 53, wherein the back flush system is fluidly coupled to the second reactor.
66. The biodigestion reactor system of any of examples 58-65, wherein the one or more products contain one or more of B, Ca, Cu, Fe, Mn, Mg, Mo, N, P, K, Na, Zn, one or more chlorides, one or more sulfates, one or more nitrates, one or more nitrites, one or more carbonates, fulvic acid, and humic acid.
67. The biodigestion reactor system of any of examples 58-66, wherein the second reactor comprises a plurality of outlets and one or more products are extracted from one or more of the plurality of outlets based on a desired composition of the product.
68. The biodigestion reactor system of any of examples 58-67, further comprising an evaporator coupled to an output of the second reactor.
69. The biodigestion reactor system of any of examples 58-68, further comprising an active species sensor located between the active species sources and the first reactor.
70. The biodigestion reactor system of any of examples 58-69, further comprising one or more sensors selected from the group consisting of an NH3 sensor, a CO2 sensor, a dissolved oxygen sensor, a pH sensor, an NOx sensor, a humidity sensor, and a temperature sensor coupled to the first reactor.

71. The biodigestion reactor system of any of examples 58-70, further comprising one or more sensors selected from the group consisting of an NH3 sensor, a CO2 sensor, a dissolved oxygen sensor, a pH sensor, an NOx sensor, a humidity sensor, and a temperature sensor coupled to the second reactor.
72. The biodigestion reactor system of any of examples 58-71, wherein biodigestion in the first reactor comprises aerobic digestion.
73. The biodigestion reactor system of any of examples 58-72, wherein biodigestion in the second reactor comprises aerobic digestion.
74. The biodigestion reactor system of any of examples 58-73, wherein the first reactor comprises a plurality of sensors in the first vessel to monitor the composition of the partially digested composition at a plurality of heights within the first vessel.
75. The biodigestion reactor system of any of examples 58-74, wherein the second reactor comprises a plurality of sensors in the second vessel to monitor the composition of the one or more products at a plurality of heights within the second vessel.
76. The biodigestion reactor system of any of examples 58-75, wherein the first reactor comprises a filter between the first input and the output of the first reactor.
77. The biodigestion reactor system of any of examples 58-76, wherein one or more of the first agitator and the second agitator comprise a venturi inductor.
78. The biodigestion reactor system of any of examples 58-77, wherein process conditions in the first reactor are designed for aerobic microorganism growth.
79. The biodigestion reactor system of any of examples 58-78, wherein process conditions in the second reactor are designed for enzyme production.
80. The biodigestion reactor system of any of examples 58-79, further comprising one or more additional biodigestion reactors fluidly coupled to one or more of the first biodigestion reactor and the second biodigestion reactor.
81. The biodigestion reactor system of any of examples 58-80, further comprising one or more breeder reactors.
82. The biodigestion reactor of any of examples 58-81, further comprising a circulation line to feed a gas between a first biodigestion reactor and a second biodigestion reactor.
83. The biodigestion reactor of any of examples 58-82, further comprising a circulation line to feed a liquid from a first or second biodigestion reactor to a grinder.

1. A biodigestion reactor system comprising:
a first reactor comprising a first vessel, a first input for receiving organic material, and one or more microorganisms within the vessel, wherein the one or more microorganisms breakdown the organic material to form a partially digested composition;
a second reactor comprising a second vessel, the second vessel fluidly coupled to the first vessel for receiving the partially digested composition from a first output of the first reactor;
a circulation loop coupled to a first output of the second reactor and a second input of the first reactor; and
a receiving tank fluidly coupled to a second output of the second reactor,
wherein a concentration of one or more products from the second reactor is manipulated by adjusting one or more of a location of the first output of the second reactor, reaction time, the microorganisms, a number of first reactors, a number of second reactors, and a rate of circulation.

2. The biodigestion reactor system of claim 1, further comprising a grinder and hopper coupled to the first input.

3. The biodigestion reactor system of claim 1, further comprising an active species source to treat the organic material prior to the organic material entering the first reactor.

4. The biodigestion reactor system of claim 1, wherein the organic material comprises material selected from one or more of the group consisting of food waste, paper, cardboard, animal waste, and other biodegradable organic material.

5. The biodigestion reactor system of claim 1, wherein the first reactor comprises one or more first reactor agitators.

6. The biodigestion reactor system of claim 1, wherein the one or more microorganisms are selected from one or more of bacteria and fungi.

7. The biodigestion reactor system of claim 1, wherein the second reactor comprises one or more second reactor agitators.

8. The biodigestion reactor system of claim 1, further comprising a back flush system for the first reactor.

9. The biodigestion reactor system of claim 8, wherein the back flush system for the first reactor is fluidly coupled to the second reactor.

10. The biodigestion reactor system of claim 5, wherein the one or more first reactor agitators comprise a pump.

11. The biodigestion reactor system of claim 1, wherein the one or more products contain one or more soil amendments.

12. The biodigestion reactor system of claim 1, wherein the one or more products contain one or more of B, Ca, Cu, Fe, Mn, Mg, Mo, N, P, K, Na, Zn, one or more chlorides, one or more sulfates, one or more nitrates, one or more nitrites, one or more carbonates, fulvic acid, and humic acid.

13. The biodigestion reactor system of claim 1, wherein a pH fluid in the second vessel ranges from about 4.8 to about 9.

14. The biodigestion reactor system of claim 1, wherein the second reactor comprises a plurality of outlets and one or more products are extracted from one or more of the plurality of outlets based on a desired composition of the one or more products.

15. The biodigestion reactor system of claim 1, further comprising an evaporator coupled to an output of the second reactor.

16. The biodigestion reactor system of claim 1, further comprising an active species sensor upstream of the first input for receiving organic material.

17. The biodigestion reactor system of claim 1, further comprising one or more sensors selected from the group consisting of NH₃, CO₂, temperature, pH, an NOₓ, a humidity, and a dissolved oxygen concentration coupled to the first reactor.

18. The biodigestion reactor system of claim 1, further comprising one or more sensors selected from the group consisting of NH₃, CO₂, temperature, pH, an NOₓ, a humidity, and a dissolved oxygen concentration coupled to the second reactor.

19. The biodigestion reactor system of claim 1, wherein biodigestion in the first reactor comprises aerobic digestion.

20. The biodigestion reactor system of claim 1, wherein biodigestion in the first reactor comprises greater than 80% aerobic digestion.

21. (canceled)

22. (canceled)
